(19) United States
(12) Patent Application Publication
(10) Pub. No.: US 2003/0187413 A1
(43) Pub. Date: Oct. 2, 2003

(54) Absorbent article having a multilayer blended core and a method of forming

(75) Inventor: David Arthur Fell, Neenah, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/108,005

(22) Filed: Mar. 26, 2002

(51) Int. Cl. A61F 13/15; A61F 13/20

(52) U.S. Cl. 604/367

(57) ABSTRACT

An absorbent core for use in an absorbent article, the absorbent article and a method of forming the absorbent core and article are disclosed. The absorbent core includes at least three absorbent layers. The first absorbent layer is formed from a stabilized material containing a superabsorbent and has a predetermined basis weight. The two remaining absorbent layers are positioned below the first absorbent layer. Each of the two remaining absorbent layers is formed from a stabilized material containing a superabsorbent and each has a basis weight which is at least equal to the basis weight of the first absorbent layer. The absorbent article includes a liquid permeable bodyside liner and a liquid-impermeable baffle enclosing the above described absorbent core. The method describes forming the absorbent layers into an absorbent core and using the absorbent core to form an absorbent article.

### BACKGROUND OF THE INVENTION

Absorbent articles such as catamenial pads, sanitary napkins, pantyliners, and the like, are designed to be worn adjacent to a woman's pudendum to absorb body fluid such as menses, blood, urine and other body excretions. It has been found that many women suffering from incontinence will buy and use a feminine care product, such as a pantyliner or a sanitary napkin, for the purpose of absorbing and retaining urine.

Incontinent users experience important differences from menstruating women and the use of commercially available feminine care products may not satisfy their specific needs. Most incontinent users require a product that can absorb and retain urine over an extended period of time. Since feminine care products are specifically designed to absorb and retain menses, many do not contain superabsorbents. Superabsorbents are capable of retaining large quantities of body fluid, such as urine, but it is known that they can impede the flow of menses. Without the presence of superabsorbents, many feminine care products do not have the fluid retention capacity needed by incontinent users. The presence of superabsorbents in incontinence products allow the urine to be locked away so the product feels dry to the wearer. Many incontinence users tend to expel only a few drops of urine at a time and therefore they tend to wear their products over a longer time period. In addition, many incontinent users are older, frugal or on a fixed income and therefore some tend to wear their products for an extended period of time in order to save money. Another reason many incontinent users wear pantyliners or ultra thin catamenial pads for incontinence is that most incontinence products are thick and bulky rather than being thin and discreet. Incontinent users have a strong psychological reason for not wanting other people to know that they suffer from incontinence.

Because of the above concerns, there is a need to produce a relatively inexpensive, thin incontinence article or pantyliner, having a thickness of less than about 15 millimeters, which can absorb and retain from about 10 g (grams) to about 1200 g of urine or more.

### SUMMARY OF THE INVENTION

Briefly, this invention relates to an absorbent core and an absorbent article. A method of forming the absorbent core and article are also disclosed. The absorbent core includes at least three absorbent layers. The first absorbent layer is formed from a stabilized material containing a superabsorbent and has a predetermined basis weight. The lower absorbent layers are positioned below the first absorbent layer. Each of the remaining absorbent layers is formed from a stabilized material containing a superabsorbent and each has a basis weight which is at least equal to the basis weight of the first absorbent layer. The absorbent article includes a liquid permeable bodyside liner and a liquid-impermeable baffle enclosing the above described absorbent core. The method describes forming the absorbent layers into an absorbent core and using the absorbent core to form an absorbent article.

### BRIEF DESCRIPTION OF THE DRAWINGS

FIG. 1 is a top view of an absorbent article such as a thin incontinence pad or a pantyliner designed to absorb and retain urine.

FIG. 2 is a cross-sectional view of the absorbent article shown in FIG. 1 taken along line 2-2 and showing the first and second absorbents forming the absorbent core.

FIG. 3 is an enlarged view of a portion of the first absorbent shown in FIG. 2.

FIG. 4 is an alternative cross-sectional view of the first and second absorbents forming the absorbent core with the second absorbent being C-folded.

FIG. 5 is another alternative cross-sectional view of the first and second absorbents forming the absorbent core with the second absorbent being cut into two distinct layers.

FIG. 6 is a schematic of a sheet of absorbent material that is folded upon itself or cut in half to form the second absorbent.

FIG. 7 is a schematic of a sheet of absorbent material that is folded upon itself three times or cut twice to form the second absorbent.

FIG. 8 is a cross-sectional view of at least one first absorbent layer positioned above an absorbent layer folded at least three times.

FIG. 9 is a cross-sectional view of at least one first absorbent layer positioned above at least four separate and distinct absorbent layers.

FIG. 10 is a cross-sectional view of at least two first absorbent layers positioned above at absorbent layer folded at least three times.

FIG. 11 is a cross-sectional view of at least two first absorbent layers positioned above at least four separate and distinct absorbent layers.

### DETAILED DESCRIPTION

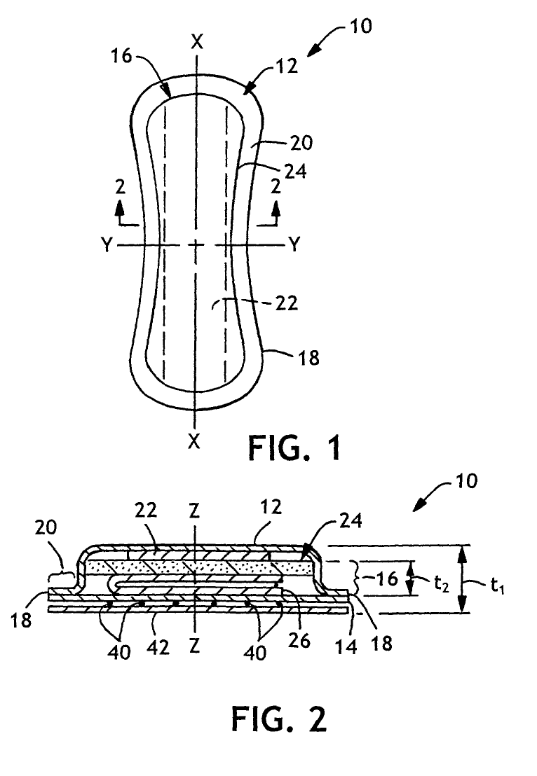

Referring to FIGS. 1 and 2, an absorbent article 10 is shown which is depicted as a thin incontinence pad or pantyliner. The absorbent article 10 is designed to be secured to an inside surface of a person's undergarment by a garment adhesive and is designed to absorb and retain urine expelled from the body. For simplicity, the term “urine” has been used, throughout, however, other bodily exudates could certainly be absorbed. The absorbent article 10 is an elongated product having a central longitudinal axis X-X, a central transverse axis Y-Y, and a vertical axis Z-Z. The absorbent article 10 is relatively thin. By “thin” it is meant that the absorbent article 10 has a thickness of less than about 15 millimeters. Desirably, the absorbent article 10 has a thickness of less than about 8 millimeters, and most desirably, the absorbent article 10 has a thickness of less than about 5 millimeters. The absorbent article 10 has a fluid retention capacity capable of absorbing from about 10 grams to about 1200 grams of urine or more. More desirably, the article can absorb 20 to 250 grams of urine. Most desirably, the absorbent article 10 will be able to absorb about 50 grams of urine.

The absorbent article 10 includes a liquid permeable liner or cover 12, a liquid-impermeable baffle 14, and an absorbent core 16 positioned and enclosed between the liner 12 and the baffle 14. The bodyside liner 12 is designed to be in contact with the wearer's body. The bodyside liner 12 can be constructed of a woven or nonwoven material that is easily penetrated by body fluid, especially urine. The liner 12 can also be formed from either natural or synthetic fibers. Suitable materials include bonded-carded webs of polyester, polypropylene, polyethylene, nylon or other heat-bondable fibers. Other polyolefins, such as copolymers of polypropylene and polyethylene, linear low-density polyethylene, finely perforated film webs and net materials, also work well. A suitable material is a soft, wettable homopolymer spunbond having a basis weight of from about 13 grams per square meter (gsm) to about 27 gsm. Another suitable material is an apertured thermoplastic film. Still another preferred material for the bodyside liner 12 is a spunbond web of polypropylene. The spunbond web can contain from about one percent (1%) to about six percent (6%) of titanium dioxide pigment to give it a clean, white appearance. When the liner 12 is constructed from a spunbond web, it is desirable to use a uniform thickness of spunbond because it will provide sufficient strength to resist being torn or pulled apart during use. The most desired polypropylene webs have a basis weight of from about 13 to about 40 grams per square meter (gsm). An optimum basis weight is from about 15 gsm to about 25 gsm.

It should be noted the bodyside liner 12 could be coated, sprayed or otherwise treated with a surfactant to make it hydrophilic. By “hydrophilic” it is meant that the bodyside liner 12 will have a strong affinity for water at a contact angle of less than 180 degrees. When the bodyside liner 12 is formed from a hydrophilic material, it will allow the body fluid to pass quickly therethrough. The bodyside liner 12 can also be embossed or dyed to improve the aesthetic appearance of the absorbent article 10.

The liquid permeable liner 12 and the liquid-impermeable baffle 14 cooperate to enclose and retain the absorbent core 16 therebetween. The liner 12 and the baffle 14 can be cut, sized and shaped to have a coterminous outer edge 18. When this is done the liner 12 and the baffle 14 can be bonded in face to face contact to form an absorbent article 10 having a peripheral seal or fringe 20. The peripheral fringe 20 can be formed to have a width of about 5 millimeters. Desirably, the liner 12 and the baffle 14 will each have a generally dogbone or hourglass configuration. With a dog bone or hourglass configuration, the absorbent article 10 will have a narrow section located adjacent to the central transverse axis y-y that separates a pair of larger, end lobes. The end lobes can be sized and/or shaped differently, if desired. An absorbent article 10 having a dogbone or hourglass shape is more comfortable to wear than a generally rectangular shaped product. The absorbent article 10 can also be asymmetrical. The liner 12 and the baffle 14 can be bonded or sealed together about their periphery by a construction adhesive to form a unitary absorbent article 10. Alternatively, the liner 12 and the baffle 14 can be bonded together by heat, pressure, by a combination of heat and pressure, by ultrasonics, etc. to form a secure attachment.

The liquid-impermeable baffle 14 can be designed to permit the passage of air or vapor out of the absorbent article 10 while blocking the passage of body fluid, such as urine. The baffle 14 can be made from any material exhibiting these properties. The baffle 14 can also be constructed from a material that will block the passage of vapor as well as fluids, if desired. A good material for the baffle 14 is a micro-embossed, polymeric film, such as polyethylene or polypropylene. Bicomponent films can also be used. A desired material is polyethylene film.

Referring to FIG. 2, the absorbent article 10 is shown having a transfer layer 22. The transfer layer 22 is optional and can be eliminated if desired. The transfer layer 22, which may contain a plurality of apertures formed therethrough, is positioned between the bodyside liner 12 and the absorbent core 16 and is aligned along the central longitudinal axis x-x. Desirably, the transfer layer 22 is positioned immediately below the bodyside liner 12 and is in direct face to face contact therewith. The transfer layer 22 can be secured, for example by using an adhesive or another attachment means, to the absorbent core 16, in order to facilitate a good transfer of body fluid therebetween. The transfer layer 22 can extend over a portion of the length of the absorbent core 16 or it can extend over the entire length of the absorbent core 16. Desirably, the transfer layer 22, when present, will extend over at least 70% of the length of the absorbent core 16. Although the transfer layer 22 is optional, when present, it does provide good fluid movement of the urine downward from the bodyside liner 12 into the absorbent core 16. This downward movement of the urine is parallel to the vertical axis z-z. The z-axis is perpendicularly arranged relative to the x and y-axes. In addition, the transfer layer 22 inhibits the flow of urine from the absorbent core 16 back up into the liner 12. This phenomenon is commonly referred to as rewet. It is important that incontinence pads and pantyliners do not exhibit rewet because the consumers do not like a wet feeling product against their skin.

The transfer layer 22 can be constructed from a material that will provide good fluid transfer. Typical materials that can be used for the transfer layer 22 are spunbond, coform and carded webs. One useful material is a wettable nonwoven having a basis weight of from about 13 gsm to about 50 gsm. The transfer layer 22 can be treated to make it hydrophilic. The transfer layer 22 can also be dyed to a different color than the color of the bodyside liner 12 and/or the absorbent core 16. A light blue, pink, or peach color has been found to be desirable, as these are pleasing colors to the ultimate consumer. The transfer layer 22 can alternatively be white in color yet will still be distinguishable from the bodyside liner 12 which may have a different shade of white. A benefit of making the transfer layer 22 a different color than the absorbent core 16 is that it presents a fluid target for the wearer.

It should be noted that the transfer layer 22 can be embossed to improve the aesthetic appearance of the absorbent article 10 since the transfer layer 22 is visible beneath the bodyside liner 12.

It is also possible to substitute a surge layer (not shown) for the transfer layer 22. The purpose of a surge layer is to quickly take up and temporarily hold the urine until the absorbent core 16 has adequate time to absorb the urine. The surge layer can be formed from various materials. Two good materials from which the surge layer can be formed include a crimped bicomponent spunbond or a bonded carded web. When a surge layer is utilized, it should be designed to have a basis weight of from about 30 gsm to about 85 gsm and a thickness ranging from about 0.15 mm to about 2 mm. The following U.S. patents teach surge layers: U.S. Pat. Nos. 5,364,382; 5,429,629; 5,490,846 and 5,486,166.

Still referring to FIG. 2, the absorbent article 10 has an absorbent core 16 that is positioned between the transfer layer 22 and the liquid-impermeable baffle 14. If no transfer layer is present, the absorbent core 16 is positioned between the bodyside liner 12 and the liquid-impermeable baffle 14. The absorbent core 16 includes a first absorbent 24 and a second absorbent 26. The first absorbent 24 is arranged close to the liner 12 and is positioned vertically above the second absorbent 26. The first absorbent 24 can be in direct face to face contact with the second absorbent 26. The first absorbent 24 can be adhered, for example, by an adhesive, to the second absorbent to ensure intimate contact and better fluid transfer therebetween. The first absorbent 24 is desirably an airlaid material. Airlaid materials are commercially available from several manufacturers. Concert GmbH is one such supplier of airlaid material that can be used to construct the absorbent article 10. Concert GmbH has an office located at Am Lehmberg 10, 16928 Falkenhagen, Germany.

Even though it is preferred that the first and second absorbents, 24 and 26 respectively, be in direct contact with one another, it is possible to place one or more layers of tissue therebetween. Some manufacturers like to wrap an absorbent which contains superabsorbent particles so as to prevent the superabsorbent particles from escaping from the finished product.

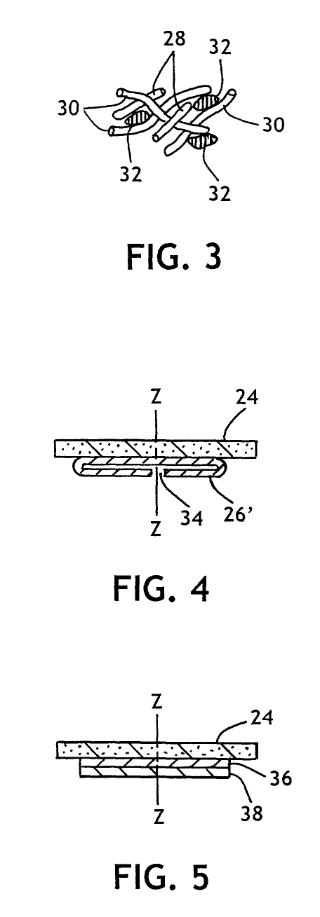

Referring now to FIG. 3, the first absorbent 24 is a stabilized material, desirably an airlaid material, constructed of a blend of a first group of fibers 28, a binder 30, desirably in the form of a second group of fibers, and a superabsorbent 32. All three components 28, 30 and 32 are cured to form a stabilized, airlaid absorbent structure. The first absorbent 24 also has a predetermined basis weight of from about 100 gsm to about 600 gsm. Desirably, the first absorbent 24 has a basis weight of from about 100 gsm to about 400 gsm. Most desirably, the first absorbent 24 has a basis weight of about 100 gsm to about 300 gsm. The first group of fibers 28 can be cellulosic fibers, such as pulp fibers, that are short in length, have a high denier, and are hydrophilic. The first group of fibers 28 can be formed from 100% softwood fibers. Desirably, the first group of fibers 28 is southern pine Kraft pulp fibers having a length of about 1.0 mm to about 3.5 mm and a denier of greater than 2. The denier of cellulosic fibers can be determined by running a coarseness test on a Kajanni analyzer to obtain a coarseness value in the units of milligrams per 100 meters (mg/100 m). This coarseness value is then divided by a constant value 11.1 to obtain a common textile denier in the units of grams per 9000 meters (g/9000 m). A suitable material to use for the first group of fibers 28 is Weyerhaeuser NB 416 pulp fibers which are commercially available from Weyerhaeuser Company. Weyerhaeuser Company has an office located at 33650 6th Avenue South, Federal Way, Wash. 98003.

Referring again to FIG. 1, the first absorbent 24 is depicted as having a shaped periphery in the form of a dog bone configuration. Other shapes, such as an hourglass shape, an oval shape, a trapezoid shape, or an asymmetrical shape formed about the longitudinal axis, etc. can also be used. A peripheral shape absorbent article having the first absorbent 24 that is narrowest in the middle along the central transverse axis Y-Y works well for it is more comfortable to wear. A trapezoidal or tapered configuration works well for a male incontinent product.

The binder portion of the first absorbent 24 can be a chemical coating. Desirably, the binder portion of the first absorbent 24 will consist of a second group of fibers 30. The second group of fibers 30 can be synthetic binder fibers. Synthetic binder fibers are commercially available from several suppliers. One such supplier is Trevira GmbH & Company KG having a mailing address of Max-Fischer-Strasse 11, 86397 Bobingen, Deutschland. Another supplier of binder fibers is Fibervisions a/s having a mailing address of Engdraget 22, Dk-6800 Varde, Denmark. A third supplier of binder fibers is KoSa having a mailing address of P.O. Box 4, Highway 70 West, Salisbury, N.C. 28145. Desirably, the second group of fibers 30 are bicomponent fibers having a polyester core surrounded by a polyethylene sheath. Alternatively, the second group of fibers 30 can be bicomponent fibers having a polypropylene core surrounded by a polyethylene sheath.

The fibers making up the second group of fibers 30 can be longer in length and have a lower denier than the fibers making up the first group of fibers 28. The length of the fibers 30 can range from about 3 mm to about 6 mm. A fiber length of 3 mm works well. The fibers 30 can have a denier of less than or equal to 2. The fibers 30 should be moisture insensitive and can be either crimped or non-crimped. Crimped fibers are desired since they process better.

The first absorbent 24 contains a superabsorbent 32. A superabsorbent is a material that is capable of absorbing at least 10 grams of water per gram of superabsorbent material. The superabsorbent 32 is desirably in the shape of small particles, although fibers, flakes or other forms of superabsorbents can also be used. A suitable superabsorbent 32 is FAVOR® 880. FAVOR® is a registered trademark of Stockhausen, Inc. having an office located at 2408 Doyle Street Greensboro, N.C. 27406. FAVOR® 880 is a commercial designation of one of Stockhausen's superabsorbents. Other similar types of superabsorbents, some of which are commercially available from Stockhausen Inc., can also be used. Desirably, the superabsorbent 32 is present in a weight percent of from about 10% to about 60%. It is important that the first absorbent 24 contain a superabsorbent to keep the user dry since it is closest to the user's body.

The individual components 28, 30 and 32 of the first absorbent 24 can be present in varying amounts. However, it has been found that the following percentages work well in forming the thin absorbent article 10. The first group of fibers 28 can range from about 30% to about 85%, by weight, of the first absorbent 24. The second group of fibers 30 can range from about 3% to about 20%, by weight, of the first absorbent 24. And the superabsorbent 32 can range from about 10% to about 60%, by weight, of the first absorbent 24. It has been found that forming a first absorbent 24 with about 58% of the first group of fibers 28, about 10% of the second group of fibers 30, and about 32% of superabsorbent works well for absorbing and retaining urine.

The first group of fibers 28 should be present in the first absorbent 24 by a greater percent, by weight, than the second group of fibers 30. By using a greater percent of the first group of fibers 28 one can reduce the overall cost of the first absorbent 24. The first group of fibers 28 also ensures that the absorbent article 10 has sufficient fluid absorbing capacity. Cellulosic fibers 28, such as pulp fibers, are generally less expensive than synthetic binder fibers 30. For good performance, the second group of fibers 30 can make up at least about three percent (3%) of the first absorbent 24, by weight to ensure that the first absorbent 24 has sufficient tensile strength. As stated above, the first absorbent 24 can be a mixture of the components 28, 30 and 32.

The first absorbent 24 is compressed in a substantially dry condition after heat curing at a temperature of about 165 degrees Celsius for a time of from about 8 seconds to about 10 seconds. The first absorbent 24 is compressed to a density ranging from about 0.09 grams per cubic centimeter g/cm³ to about 0.3 g/cm³. Desirably, the first absorbent 24 is compressed in a substantially dry condition to a density ranging from about 0.15 g/cm³ to about 0.22 g/cm³. Most desirably, the first absorbent 24 is compressed in a substantially dry condition to a density of at about 0.2 g/cm³. This compression of the first absorbent 24 will assist in forming the thin absorbent article 10.

It is important to note that the stabilized material, making up the first and second absorbents, 24 and 26 respectively, should have sufficient tensile strength in the machine direction to allow winding it into rolls which can later be unwound and processed on converting equipment. Sufficient tensile strength can be achieved by varying the content of the binder fiber, adjusting the curing conditions, changing the specific density to which the fibers are compacted, as well as other ways known to one skilled in the art. It has been found that the first and second absorbents, 24 and 26 respectively, should have a tensile strength of at least 12 Newtons per 50 mm (N/50 mm). Desirably, the first and second absorbents, 24 and 26 respectively, should have a tensile strength of at least 18 N/50 mm. More desirably, the first and second absorbents, 24 and 26 respectively, should have a needed tensile strength of at least 25 N/50 mm. The tensile strength of the material can be tested using a tester, model MTS/Sintech 1/S which is commercially sold by MTS Systems Corporation having a mailing address of P.O. Box 14226, Research Triangle Park, N.C. The tensile strength at peak load for the purpose of this invention is measured by securing a 50 mm wide strip of stabilized material between two movable jaws of a tensile tester. A distance of about 10 cm will initially separate the two jaws. The two jaws are then moved outward away from one another at a rate of 25 cm/minute until the strip of material breaks. The tensile strength is recorded as peak load.

Referring again to FIG. 2, the second absorbent 26 is arranged near the baffle 14 and is positioned vertically below the first absorbent 24. The second absorbent 26 can have a length that equals the length of the first absorbent 24 but desirably is sized to be slightly shorter than the length of the first absorbent 24. Most desirably, the second absorbent 26 will have a length that ranges from about 60% to about 95% of the length of the first absorbent 24. The width of the second absorbent 26 can be less than, equal to or be greater than the width of the first absorbent 24. Desirably, the width of the second absorbent 26 is equal to or less than the width of the first absorbent 24. Most desirably, the width of the second absorbent 26 is less than the width of the first absorbent 24. Even more desirably, the width of the second absorbent 26 is from about 40% to about 75% of the width of the first absorbent 24.

The second absorbent 26 is also a stabilized material, desirably an airlaid material, constructed of a blend of a first group of fibers 28, a binder 30, desirably in the form of a second group of fibers, and a superabsorbent 32. Desirably, the first and second absorbents, 24 and 26 respectively, are of similar composition. The compositions of the first and second absorbents, 24 and 26 respectively, can be identical, if desired. The second absorbent 26 has a basis weight which is equal to or greater than the basis weight of the first absorbent 24. By so constructing the second absorbent 26, one can be assured that the absorbent article 10 will have adequate absorbent capacity to function properly. Desirably, the basis weight of the second absorbent 26 is greater than the basis weight of the first absorbent 24. More desirably, the basis weight of the second absorbent 26 is at least 2 times greater than the basis weight of the first absorbent 24. The size, area, shape, etc. of the second absorbent 26 can be adjusted, such as by folding, slitting, cutting, using one or more layers, etc. to achieve the desired absorbent capacity.

It should be noted that when the first absorbent 24 is die cut into a shaped configuration, such as a dog bone shape, a certain amount of waste is created. Such waste will increase the cost to manufacture the absorbent article 10. Therefore, it is desirable to utilize a minimum basis weight for the first absorbent 24 in order to reduce waste. Since the second absorbent 26 has a generally rectangular configuration, it is more economical to increase the absorbent capacity of the absorbent article 10 by adjusting the area and/or the number of layers from which the second absorbent 26 is constructed.

Still referring to FIG. 2, one way of achieving the desired basis weight of the second absorbent 26 is depicted. In this figure, the second absorbent 26 is longitudinally folded upon itself into a U-shaped configuration. This folding of the second absorbent 26 doubles its basis weight.

Referring to FIGS. 4 and 5, two alternative ways to increase the basis weight of the second absorbent 26 while using the same material as was used for the first absorbent 24 are depicted. In FIG. 4, a second absorbent 26′ is C-folded so as to have a channel or slit 34 formed which extends the length of the second absorbent 26′ and which is aligned with the vertical axis z-z. The channel or slit 34 can face towards the user or away from the user. In FIG. 5, a third way of increasing the basis weight of a second absorbent is depicted while using a similar composition as was used for the first absorbent 24. In FIG. 5, the second absorbent is shown as two separate and distinct layers 36 and 38. The two layers 36 and 38 together will have a greater basis weight than the first absorbent 24 since all are formed from a similar composition or from the identical material.

It should be noted that if one desired to manufacture an absorbent article 10 having a lower fluid absorbent capacity, then one could construct the second absorbent 26 as a single layer.

The second absorbent 26 has a predetermined basis weight ranging from about 200 gsm to about 1200 gsm. Desirably, the second absorbent 26 has a basis weight of from about 300 gsm to about 600 gsm. Most desirably, the second absorbent 26 has a basis weight of about 400 gsm. Depending on the required absorbent capacity one wished for the finished absorbent article 10, the second absorbent 26 could be constructed to have a basis weight that is a whole number multiple, or an integral multiple, of the first absorbent 24.

As stated above, the first group of fibers 28 making up the second absorbent 26 can be cellulosic fibers, such as pulp fibers. The fibers within the first group of fibers 28 can be short in length, have a high denier, and are hydrophilic. The first group of fibers 28 can be formed from 100% softwood fibers. Desirably, the first group of fibers 28 is southern pine Kraft pulp fibers having a length of about 1.0 mm to about 3.5 mm. The fibers 28 can have a denier of greater than 2. The denier of cellulosic fibers can be determined as stated previously. A suitable material to use for the first group of fibers 28 is Weyerhaeuser NB 416 pulp fibers.

Referring again to FIG. 2, the second absorbent 26 is depicted as being narrower in width than the first absorbent 24. The second absorbent 26 can have a generally rectangular configuration or some other shape, if desired. The reason for using the generally rectangular configuration is that some stabilized material, especially airlaid material, are relatively difficult to recycle. By forming the second absorbent 26 into a generally rectangular shape, one can minimize waste during the manufacturing process and produce a lower cost absorbent article 10. In addition, by constructing the first and second absorbents, 24 and 26 respectively, out of the same or similar composition, one can reduce one's inventory of raw materials and thereby reduce the cost required to manufacture the absorbent article 10. Desirably, the dimensions of the first and second absorbents, 24 and 26 respectively, can be selected so that the slit width of the supply rolls from which the material is cut are the same. This will further reduce inventory complexity.

The binder portion of the second absorbent 26 can be a chemical coating. Desirably, the binder portion of the second absorbent 26 will consist of a second group of fibers 30. The second group of fibers 30 is desirably synthetic binder fibers identical to those used to form the first absorbent 24. Desirably, the second group of fibers 30 is bicomponent fibers having a polyester core surrounded by a polyethylene sheath. Alternatively, the second group of fibers 30 is bicomponent fiber having a polypropylene core surrounded by a polyethylene sheath.

The fibers making up the second group of fibers 30 can be longer in length and have a lower denier than the fibers making up the first group of fibers 28. The length of the fibers 30 can range from about 3 mm to about 6 mm. A fiber length of 3 mm works well. The fibers 30 can have a denier equal to or less than 2. The fibers 30 can be moisture insensitive and can be either crimped or non-crimped. Crimped fibers are desired since they are commercially available and sometimes they are easier to process.

The second absorbent 26 also contains a superabsorbent 32. As explained above, a superabsorbent is a material capable of absorbing at least 10 grams of water per gram of superabsorbent material. The superabsorbent 32 is desirably in the shape of small particles, although fibers, flakes or other forms of superabsorbents can also be used. The superabsorbent 32 used in the second absorbent 26 can be identical in composition to the superabsorbent used in the first absorbent 24. A suitable superabsorbent 32 is FAVOR® 880. FAVOR® 880 is a registered trademark of Stockhausen, Inc. having an office located at 2408 Doyle Street Greensboro, N.C. 27406. Other similar types of superabsorbents, some of which are commercially available from Stockhausen Inc., can also be used. Desirably, the superabsorbent 32 is present from about 10 weight percent to about 60 weight percent of the stabilized material.

The individual components 28, 30 and 32 of the second absorbent 26 can be present in varying amounts. However, it has been found that the following percentages work well in forming the thin absorbent article 10. The first group of fibers 28 can range from about 30% to about 85%, by weight, of the second absorbent 26. The second group of fibers 30 can range from about 3% to about 20%, by weight, of the second absorbent 26. And the superabsorbent 32 can range from about 10 to about 60%, by weight, of the second absorbent 26. It has been found that forming a second absorbent 26 with about 58% of the first group of fibers 28, about 10% of the second group of fibers 30, and about 32% of superabsorbent works well for absorbing and retaining body fluid, especially urine.

The first group of fibers 28 should be present in the second absorbent 26 by a greater percent, by weight, than the second group of fibers 30 so as to reduce the overall cost of the second absorbent 26. Cellulosic fibers 28, such as pulp fibers, are generally less expensive than synthetic binder fibers 30. For good performance, the second group of fibers 30 should make up at least about three percent (3%) of the second absorbent 26, by weight to ensure sufficient tensile strength. As stated above, the second absorbent 26 should be a mixture of the components 28, 30 and 32.

Like the first absorbent 24, the second absorbent 26 is compressed in a substantially dry condition after heat curing at a temperature of about 165 degrees Celsius for a time of from about 8 seconds to about 10 seconds. The second absorbent 26 is compressed to a density ranging from about 0.09 grams per cubic centimeter g/cm³ to about 0.3 g/cm³. Desirably, the second absorbent 26 is compressed in a substantially dry condition after heat curing as explained above to a density ranging from about 0.15 g/cm³ to about 0.22 g/cm³. Most desirably, the second absorbent 26 is compressed in a substantially dry condition after heat curing as explained above to a density of about 0.2 g/cm³. This compression of the second absorbent 26 will assist in forming the thin absorbent article 10.

It should be noted that the first and second absorbents, 24 and 26 respectively, are desirably compressed to the same density during the manufacturing process.

Referring back to FIG. 2, the absorbent article 10 is shown having a thickness t₁ of less than about 15 mm. Desirably, the absorbent article 10 has a thickness t₁ of from about 5 mm to about 8 mm. More desirably, the absorbent article 10 has a thickness t₁ of about 5 mm. The thickness t₁ or caliper of the absorbent article 10 can be determined by measuring the thickness t₁ of the absorbent article 10 with a bulk tester such as a Digimatic Indicator Gauge, type DF 1050E which is commercially available from Mitutoyo Corporation of Japan. Typical bulk testers utilize a smooth platen that is connected to the indicator gauge. The platen has dimensions that are smaller than the length and width of the second absorbent 26. The thickness of the absorbent article 10 is measured under a pressure of 0.35 kPa.

Still referring to FIG. 2, the absorbent core 16 also has a thickness t₂ of less than about 14 mm. Desirably, the absorbent core 16 has a thickness t₂ ranging from about 4 mm to about 13 mm. More desirably, the absorbent core 16 has a thickness t₂ of less than about 12 mm. The thickness t₂ of the absorbent core 16 can be measured in a similar fashion as the thickness t₁ of the absorbent article 10 except that the absorbent core 16 will first be removed from the absorbent article 10.

The absorbent article 10 further is shown having a garment adhesive 40 secured to an exterior surface of the baffle 14. The garment adhesive 40 can be a hot melt, pressure sensitive adhesive that functions to attach the absorbent article 10 to the inner crotch portion of an undergarment during use. The garment adhesive 40 enables the absorbent article 10 to be properly aligned and retained relative to the user's urethra so that maximum protection from the involuntary loss of urine can be obtained. The garment adhesive 40 can be slot coated onto the baffle 14 as one or more strips or it can be applied as a swirl pattern. The composition of the garment adhesive 40 is such that it will allow a user to remove the absorbent article 10 and reposition the article 10 in the undergarment if needed. A suitable garment adhesive 40 that can be used is Code Number 34-5602 which is commercially available from National Starch and Chemical Company. National Starch and Chemical Company has an office located at 10 Finderne Avenue, Bridgewater, N.J. 08807.

In order to protect the garment adhesive 40 from contamination prior to use, a releasable peel strip 42 is utilized. The peel strip 42 can be formed from paper or treated paper. A standard type of peel strip 42 is a white Kraft peel paper coated on one side so that it can be easily released from the garment adhesive 40. The user removes the peel strip 42 just prior to attaching the absorbent article 10 to the inner crotch portion of his or her undergarment. Three suppliers of the peel strips 42 include Tekkote, International Paper Release Products, and Namkyung Chemical Ind. Co., Ltd. Tekkote has an office located at 580 Willow Tree Road, Leonia, N.J. 07605. International Paper Release Products has an office located at 206 Garfield Avenue, Menasha, Wis. 54952. Namkyung Chemical Ind. Co., Ltd. has an office located at 202-68 Songsan-ri, Taean-eup, Hwaseoung-kum, Kyunggi, Korea. For absorbent articles, such as diapers, training pants, adult briefs and undergarments, garment adhesives are not required.

The above description teaches the use of a stabilized material, such as airlaid, for the first and second absorbents, 24 and 26 respectively. However, the unique idea of using multiple plies of the same material will work for any material that possesses sufficient tensile strength to make it through the manufacturing and/or converting processes. The same economic principles of minimizing waste from the shaped first absorbent 24 while maintaining superabsorbent in that layer apply. Examples of other materials include using wet laid webs constructed of pulp fibers and superabsorbents. Examples of these materials are described in U.S. Pat. No. 5,651,862 issued to Anderson et al. Another material commercially sold by Rayonier Inc. of Jesup, Georgia is a high-density superabsorbent containing a non-stabilized web formed on tissue, which can be slit, folded and processed on a converting line. U.S. Pat. No. 5,916,670 issued to Tan et al. teaches this material. A third material is MegaThin® a high superabsorbent containing composite produced by JATI (Japan Absorbent Technology Institute). MEGATHIN is a registered trademark of JATI. Additionally, foams, various nonwoven materials impregnated with a superabsorbent, as well as other high absorbency materials supplied as web can be used.

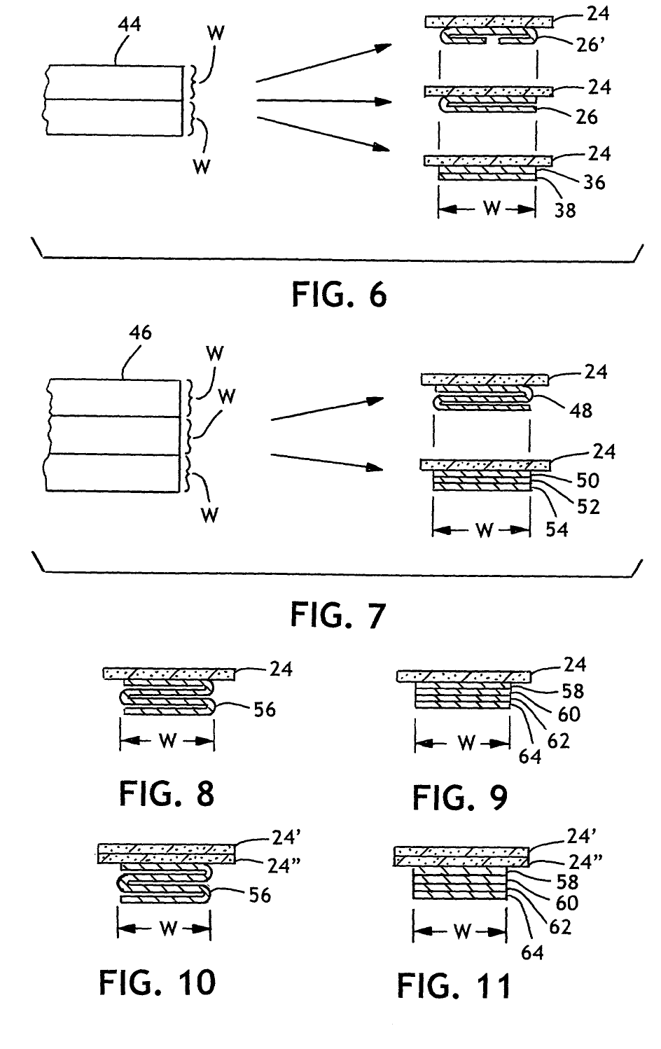

Referring now to FIG. 6, a schematic is illustrated for forming an absorbent core having at least three absorbent layers. The absorbent core has at least one first absorbent layer 24 and at least two lower or second absorbent layers. The lower or second absorbent layers can be formed from an absorbent sheet or web 44, having a width of (2w). The absorbent sheet or web 44 is folded or cut to form the second or lower absorbent layers 26, 26′ or 36 and 38. Alternatively, each of the second absorbent layers could be formed from a separate absorbent sheet. The absorbent sheet 44 can be C-folded to a width (w) to form a folded absorbent layer 26′. The C-folded absorbent layer 26′ is viewed, for purposes of this invention, as being equivalent to the two horizontally aligned layers 36 and 38. The folded absorbent layer 26′ is aligned and positioned below the first absorbent 24 to form an absorbent core. Desirably, at least a portion of the second absorbent layer 26′ is positioned directly below and in contact with the first absorbent 24.

The first absorbent 24 is at least one absorbent layer. The first absorbent 24 could be formed from a blend of fibers such as a first group of short, high denier hydrophilic fibers. The first absorbent 24 has a basis weight of from about 100 gsm to about 600 gsm. The first absorbent 24 contains from about 30% to about 85% cellulose fibers, from about 5% to about 20% binder fibers and from about 10% to about 60% superabsorbent.

The folded or second absorbent layer 26′ includes at least one layer that is folded at least once to produce the equivalent of at least two layers. Desirably, the C-folding doubles the basis weight of the second absorbent 26′. The second absorbent layer 26′ can be formed from a second group of longer, lower denier, moisture insensitive crimped synthetic fibers. The second group of fibers should make up at least about 3% of the absorbent core, by weight. The second group of fibers is desirably synthetic, long low denier binder fibers identical to those used in the first absorbent 24. The second absorbent 26′ contains from about 30% to about 85% cellulose fibers, from about 3% to about 20% binder fibers and from about 10% to about 60% superabsorbent. The second absorbent layer 26′ has a basis weight of from about 200 gsm to about 1,200 gsm. Desirably, the second absorbent 26′ has a basis weight of from about 200 gsm to about 600 gsm.

The first absorbent 24 and the second absorbent 26′ form the absorbent core. The composition, characteristics and functionality of the absorbent core can be as described above with reference to FIGS. 1-5. For example, the absorbent core can have a fluid retention capacity of from about 20 grams to about 250 grams or more. Desirably, the fluid retention capacity of the absorbent core can be about 50 grams for pantyliner type products.

It should be noted that the binder used in the second group of fibers could include binder fibers having a length of from about 3 mm to about 6 mm. Furthermore, the second absorbent 26′ should have a basis weight that is at least 2 times greater than the basis weight of the first absorbent 24. Desirably, the second absorbent 26′ should have a basis weight that is at least 2 times greater than the basis weight of the first absorbent 24. More desirably, the second absorbent 26′ should have a basis weight that is a whole number multiple of the basis weight of the first absorbent 24.

The first and second absorbents 24 and 26′ will form the absorbent core. The individual layers 24 and 26′ can be compressed before being assembled into the absorbent core or the absorbent core can be compressed after being assembled. In either case, the individual layers 24 and 26′ or the absorbent core can be compressed in a substantially dry condition to a density of at least about 0.09 g/cm³. The absorbent core is then positioned between a liquid permeable bodyside liner 12 and a liquid-impermeable baffle 14, see FIG. 2, to form an absorbent article 10.

Alternatively, the absorbent sheet 44 can be U-folded upon itself to a width (w) to form the second absorbent 26. The U-folded absorbent layer 26′ is viewed, for purposes of this invention, as being equivalent to the two horizontally aligned layers 36 and 38. Desirably, the U-folding doubles the basis weight of the second absorbent 26. The second absorbent 26 is then aligned and positioned below the first absorbent 24 to form an absorbent core. Still another alternative is to cut the absorbent sheet 44 longitudinally into two elongated strips or layers 36 and 38, each having a width (w). The two separate and distinct strips or layers 36 and 38 can be vertically aligned relative to each other and arranged below the first absorbent 24 to form an absorbent core. The two strips 36 and 38 doubles the basis weight of the absorbent sheet 44. As shown in FIG. 6, the combined basis weight of the two layers 36 and 38 is double the basis weight of the first absorbent layer 24.

Referring to FIG. 7, another schematic is illustrated for forming an absorbent core having at least four absorbent layers. The absorbent core has at least one first absorbent layer 24 and at least three second absorbent layers. The second absorbent layers can be formed from an absorbent sheet or web 46, having a width (3 w), which is folded or cut to form the second absorbent layers 48, or 50, 52 and 54. The absorbent sheet 46 can be folded twice upon itself to a width (w) to form an S-folded second or lower absorbent layer 48. The S-folded absorbent layer 48 is viewed, for purposes of this invention, as being equivalent to the three horizontally aligned layers 50, 52 and 54. Desirably, the S-folding triples the basis weight of the second absorbent 48. The second absorbent 48 is then aligned below the first absorbent 24 to form an absorbent core. Desirably, at least a portion of the second or lower absorbent 48 is positioned directly below and in contact with the first absorbent 24. The first absorbent 24 is at least one absorbent layer formed of a blend of fibers such as a first group of short, high denier hydrophilic fibers. The first absorbent 24 has a basis weight of from about 100 gsm to about 600 gsm. The second absorbent 48 consists of at least one layer folded at least twice to produce the equivalent of at least three layers. The second absorbent 48 can be formed from a second group of longer, lower denier, moisture insensitive crimped synthetic fibers. The second group of fibers should make up at least about 3% of the absorbent core, by weight. The second or lower absorbent layer(s) 48 has a basis weight of from about 300 gsm to about 1200 gsm. Desirably, the second absorbent 48 has a basis weight of from about 300 gsm to about 600 gsm. The second group of fibers can be a stabilized material such as an airlaid constructed of cellulosic fibers, a binder and a superabsorbent

It should be noted that the binder used in the second group of fibers could include binder fibers having a length of from about 3 mm to about 6 mm. Furthermore, the second absorbent 48 should have a basis weight that is at least 3 times greater than the basis weight of the first absorbent 24. Desirably, the second absorbent 48 should have a basis weight that is a whole number multiple of the basis weight of the first absorbent 24.

The first and second absorbents 24 and 48 will form the absorbent core. The individual layers can be compressed before being assembled into the absorbent core or the absorbent core can be compressed after being assembled. The absorbent layers or the absorbent core can be compressed in a substantially dry condition to a density of at least about 0.09 g/cm³. The absorbent core is then positioned between a liquid permeable bodyside liner 12 and a liquid-impermeable baffle 14, see FIG. 2, to form an absorbent article 10.

Alternatively, the absorbent sheet 46 can be cut longitudinally into three elongated strips or layers 50, 52 and 54 each having a width (w). The three separate and distinct strips or layers 50, 52 and 54 can be vertically aligned relative to one another and arranged below the first absorbent 24 to form an absorbent core. The three layers 50, 52 and 54 will have a basis weight which is triple the basis weight of the absorbent sheet 46. The upper most absorbent layer 50 should be in direct contact with the first absorbent layer 24. The three absorbent layers 50, 52 and 54 can be of identical or similar composition. The composition, characteristics and functionality of the different absorbent layers can be as described above with reference to FIGS. 1-6.

Referring now to FIGS. 8 and 9, two alternative cross-sectional views are depicted for constructing an absorbent core having at least five absorbent layers. In FIG. 8, an absorbent sheet is folded three times upon itself to a width (w) to form a folded second or lower absorbent 56. The folded absorbent 56 is viewed, for purposes of this invention, as being equivalent to the four horizontally aligned layers 58, 60, 62 and 64. At least a portion of the second absorbent 56 is then aligned and positioned directly below the first absorbent 24 to form an absorbent core. Desirably, a portion of the second absorbent 56 is in direct contact with the first absorbent 24. The combined basis weight of the second absorbent 56 can be equal to or be greater than the basis weight of the first absorbent 24. Most desirably, the combined basis weight of the second absorbent 56 is four times the basis weight of the first absorbent 24.

In FIG. 9, one or more absorbent sheets are cut into four elongated strips or layers 58, 60, 62 and 64 each having a width (w). The four separate and distinct strips or layers 58, 60, 62 and 64 can be vertically aligned relative to one another and arranged below the first absorbent 24 to form an absorbent core. The upper layer 58 can be in direct contact with the first absorbent 24. The combined basis weight of the second absorbent 56 can be equal to or be greater than the basis weight of the first absorbent 24. Most desirably, the combined basis weight of the second absorbent 56 is four times the basis weight of the first absorbent 24.

The four absorbent layers 58, 60, 62 and 64 can be of identical or similar composition. The composition, characteristics and functionality of the different absorbent layers can be as described above with reference to FIGS. 1-7.

Referring to FIGS. 10 and 11, two alternative cross-sectional views are depicted for forming an absorbent core. In FIG. 10, the first or upper absorbent is formed from at least two separate and distinct layers 24′ and 24″. The two layers 24′ and 24″ are vertically aligned relative to each other. The second or lower absorbent 56 is folded three times upon itself to a width (w) to form a multi-folded second absorbent. The folded second absorbent 56 is viewed, for purposes of this invention, as being equivalent to four horizontally aligned layers. The second absorbent 56 is then aligned below the first two absorbent layers 24′ and 24″ to form an absorbent core. The absorbent core will then have a total of six absorbent layers or the equivalent thereof. The combined basis weight of the second absorbent 56 can be equal to or be greater than the basis weight of the first absorbent layers 24′ and 24″.

In FIG. 1, the first absorbent is formed from at least two separate and distinct layers 24′ and 24″. The first two absorbent layers 24′ and 24″ are vertically aligned relative to each other. The second or lower absorbent will consist of at least four absorbent layers 58, 60, 62 and 64. The four absorbent layers 58, 60, 62 and 64 can be formed from one or more absorbent sheets. For example, a single absorbent sheet having a width (4w) can be cut into four elongated strips or layers 58, 60, 62 and 64 each having a width (w). The four separate and distinct strips or layers 58, 60, 62 and 64 can be vertically aligned relative to one another and arranged below the first two absorbent layers 24′ and 24″ to form an absorbent core. The combined basis weight of the four absorbent layers 58, 60, 62 and 64 can be equal to or be greater than the basis weight of the first absorbent layers 24′ and 24″. The four absorbent layers 58, 60, 62 and 64 can be of identical or similar composition. In FIG. 11, the absorbent core is constructed from six separate and distinct layers.

In the case of the embodiments shown in FIGS. 10 and 11, the combined basis weight of the second absorbent 56 can be an integral or an integral plus a half (1.5, 2.5, 3.5 times, etc.) multiple of the first absorbent.

Method

Various methods of manufacturing the absorbent article 10 will now be described. One method includes the steps of forming the first absorbent 24 from a stabilized material, such as an airlaid. The airlaid will contain a superabsorbent and have a predetermined basis weight. The airlaid material can be a rectangular, elongated strip having a transverse width of about 30 mm to about 100 mm or more. The airlaid can be unwound from a supply roll and be fed into a cutter that can cut the elongated strip into individual members. The periphery of the first absorbent 24 can be cut, for example by a die cutter, into a shaped configuration, such as a dog bone shape, an hourglass shape, an oval shape, etc.

The method further includes the step of forming the second absorbent 26 from a similar or identical stabilized material. Desirably, the stabilized material will also be an airlaid containing superabsorbent and having a predetermined basis weight. Desirably, the first and second absorbents, 24 and 26 respectively, will be formed from the same airlaid material. The airlaid can be a generally rectangular, elongated strip having a transverse width of about 30 mm to about 100 mm or more. Desirably, the second absorbent will be formed from a strip of stabilized material having the same or less width as was used to form the first absorbent 24. The airlaid can be unwound from a supply roll and doubled in thickness in at least three different ways in order to increase the basis weight of the second absorbent 26. One way is to C-fold each individual segment into a generally rectangular shape. The C-fold can have a channel or slit 34 that is located adjacent to the top or the bottom of the C-fold. A second way is to fold each individual segment upon itself into a U-shape when viewed on its side. The opening of the U-shape can face either to the left or to the right. A third way is to slice or slit each individual segment longitudinally into two or more strips. The strips are then placed one on top of the other. Regardless of the method used to form the second absorbent 26, the second absorbent 26 desirably has a width that is less than, equal to or greater than the narrowest width of the first absorbent 24. The second absorbent 26, after being doubled in thickness, can be routed to a cutter. The cutter can consist of a knife roll and a cooperating anvil roll. Here, the airlaid material is cut into individual rectangular segments.

The second absorbent 26 will have a basis weight which is at least equal to, and desirably, greater than the basis weight of the first absorbent 24. The reason for this is that by controlling the basis weight of the second absorbent 26, one can be assured that the second absorbent 26 will be able to retain at least an equal, if not greater, quantity of body fluid than the first absorbent 24. By retaining a majority of the body fluid in the second or lower absorbent 26, which is located away from the body of the wearer, the first absorbent 24 will be drier. Additional, this feature creates an absorbent article 10 that minimizes design waste by minimizing trim waste associated with cutting the first absorbent 24 into a non-rectangular shape.

Regardless of which of the three options for forming the second absorbent 26 are utilized, the method then includes the step of aligning the second absorbent 26 beneath and in direct contact with the first absorbent 24. When the second absorbent 26 is slit into two or more individual layers, the layers are vertically positioned relative to one another and below the first absorbent 24. Desirably, the second absorbent 26 is narrower in width than the width of the first absorbent 24. More desirably, the second absorbent 26 will have a smaller surface area than the first absorbent 24. Construction adhesive can be used between the first and second absorbents, 24 and 26 respectively, if needed.

It should be noted that the airlaid material could be initially, longitudinally slit into equal widths, for example about 65 mm. The first and second absorbents, 24 and 26 respectively, would then be formed from these similar width elongated strips. This would simplify production since only one specific airlaid material has to be made. Ideally, forming the absorbent core 16 out of a single airlaid material would simplify the supply chain and further reduce manufacturing cost.

The absorbent core 16, formed from the first and second absorbents, 24 and 26 respectively, is then combined with a liquid permeable liner 12 and a liquid-impermeable baffle 14 to form an absorbent article 10. A transfer layer 22 can be included in the assembling process, if desired.

The above procedure can be followed using one or more layers to form the first absorbent 24 and using two, three, four or more layers to form the second absorbent, see FIGS. 6-11. Remember that the second absorbent material 26, 26′, 48 or 56 can be folded two or more times to form the equivalent of three or more horizontal layers. The basis weight of the second absorbent could be at least two, three, four or more times the basis weight of the first absorbent 24 or 24′ and 24″. The first absorbent 24 can also be folded once to form the equivalent of two layers if one did not desire to utilize two separate layers.

It should also be noted that the basis weight of the first absorbent 24 could range from about 100 gsm to about 600 gsm. The basis weight of the second absorbent 56 can range from about 200 gsm to about 1200 gsm. Desirably, the basis weight of the second absorbent 56 or the absorbent layers 58, 60, 62 and 64 can range from about 200 gsm to about 600 gsm. Furthermore, the combined basis weight of the various absorbent layers making up the second absorbent 56 can be at least 2 times greater than the basis weight of the first absorbent 24 or the two first absorbent layers 24′ and 24″. Furthermore, an absorbent core formed according to the construction shown in FIGS. 6-11 should have a fluid retention capacity of from about 10 grams to about 1200 grams. More desirably the fluid retention capacity should be from 20 to about 250 grams. Desirably, the fluid retention capacity of the absorbent core should be about 50 grams.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

I claim:

1. An absorbent core for an absorbent article, said absorbent core comprising at least three absorbent layers, a first of said at least three absorbent layers being formed from a stabilized material containing a superabsorbent and having a predetermined basis weight, and said two remaining absorbent layers being positioned below said first absorbent layer, each of said two remaining absorbent layers being formed from a stabilized material containing a superabsorbent and each having a basis weight which is at least equal to said basis weight of said first absorbent layer.

2. The absorbent core of claim 1 wherein each of said two remaining absorbent layers has a basis weight of from about 100 gsm to about 600 gsm.

3. The absorbent core of claim 2 wherein each of said two remaining absorbent layers has a thickness of from about 3 millimeters to about 6 millimeters.

4. The absorbent core of claim 1 wherein each of said two remaining absorbent layers is a separate and distinct layer.

5. The absorbent core of claim 1 wherein said two remaining absorbent layers have a combined basis weight which is at least equal to two times the basis weight of said first absorbent layer.

6. The absorbent core of claim 1 wherein each of said two remaining absorbent layers is formed by folding an absorbent sheet at least twice upon itself.

7. The absorbent article of claim 1 wherein said absorbent core has a fluid retention capacity of from about 10 grams to about 1200 grams.

8. The absorbent core of claim 1 wherein said absorbent core includes at least four absorbent layers, a first of said at least four absorbent layers being formed from a stabilized material containing a superabsorbent and having a predetermined basis weight, and said three remaining absorbent layers are being positioned below said first absorbent layer and have a combined basis weight which is at least equal to three times said basis weight of said first absorbent layer.

9. The absorbent core of claim 8 wherein said three remaining absorbent layers are formed by folding an absorbent sheet at least two times upon itself.

10. An absorbent article, comprising:
a) a liquid permeable liner;
b) a liquid-impermeable baffle; and
c) an absorbent core having at least four absorbent layers, a first of said at least four absorbent layers being positioned between said liner and said baffle and being formed from a stabilized material containing a superabsorbent and having a predetermined basis weight, and at least three of said remaining absorbent layers being positioned between said first absorbent layer and said baffle, each of said at least three remaining absorbent layers being formed from a stabilized material containing a superabsorbent and each having a basis weight which is at least equal to said basis weight of said first absorbent layer.

11. The absorbent article of claim 10 wherein said absorbent core has a fluid retention capacity of from about 10 grams to about 1200 grams.

12. The absorbent article of claim 11 wherein said absorbent core has a fluid retention capacity of about 50 grams.

13. The absorbent article of claim 10 wherein said first absorbent layer has a basis weight of from about 100 gsm to about 600 gsm.

14. The absorbent article of claim 10 wherein each of said at least three remaining absorbent layers positioned between said first absorbent layer and said baffle has a basis weight of from about 100 gsm to about 400 gsm.

15. The absorbent article of claim 10 wherein said at least three remaining absorbent layers have a combined basis weight which is at least equal to three times said basis weight of said first absorbent layer.

16. The absorbent article of claim 10 wherein said first absorbent layer contains from about 30% to about 85% cellulosic fibers, from about 3% to about 20% binder fibers, and from about 10% to about 60% superabsorbent.

17. The absorbent article of claim 10 wherein each of said at least three remaining absorbent contains from about 30% to about 85% cellulosic fibers, from about 3% to about 20% binder fibers, and from about 10% to about 60% superabsorbent.

18. The absorbent article of claim 10 wherein said at least three remaining absorbent layers have a combined basis weight which is a whole number multiple of said basis weight of said first absorbent.

19. A method of forming an absorbent core comprising the steps of:
a) forming a first absorbent into a shaped configuration from an elongated strip of a stabilized material having a desired thickness, said stabilized material containing a superabsorbent, and said first absorbent having a predetermined basis weight;
b) forming a second absorbent from an elongated strip of stabilized material, said second absorbent including at least three layers having a combined thickness greater than said thickness of said first absorbent, said stabilized material containing a superabsorbent, and said second absorbent having a basis weight which is greater than the basis weight of said first absorbent; and
c) positioning said second absorbent directly below and in contact with said first absorbent to form said absorbent core.

20. The method of claim 19 wherein said stabilized material used to form said first and second absorbents is the same composition.

21. The method of claim 19 wherein said second absorbent comprises at least four layers.

22. The method of claim 19 wherein said second absorbent is folded at least twice to form the equivalent of three horizontal layers.

23. The method of claim 19 wherein said second absorbent is folded at least three times to form the equivalent of four horizontal layers.

24. The method of claim 19 wherein said first absorbent comprises at least two layers.

25. The method of claim 19 wherein said second absorbent comprises at least five layers.

26. The method of claim 19 wherein said second absorbent has a basis weight which is at least two times the basis weight of said first absorbent.

27. The method of claim 19 wherein said second absorbent has a basis weight which is at least three times the basis weight of said first absorbent.

28. The method of claim 19 wherein said first and second absorbents comprise six separate and distinct layers.

29. A method of forming an absorbent article comprising the steps of:
a) forming a first absorbent into a shaped configuration from an elongated strip of a stabilized material, said stabilized material containing a superabsorbent, and said first absorbent having a predetermined basis weight;
b) forming a second absorbent from an elongated strip of stabilized material, said stabilized material containing a superabsorbent, said second absorbent including at least four layers having a combined basis weight which is greater than the basis weight of said first absorbent;
c) positioning said second absorbent directly below and in contact with said first absorbent to form said absorbent core; and
d) enclosing said absorbent core between a liquid permeable liner and a liquid-impermeable baffle.
